(19) United States
(12) Patent Application Publication
(10) Pub. No.: US 2022/0133403 A1
(43) Pub. Date: May 5, 2022

(54) SYSTEMS AND METHODS FOR ABLATION USING NON-ADJACENT BIPOLES

(71) Applicant: St. Jude Medical, Cardiology Division, Inc., St. Paul, MN (US)

(72) Inventors: Eric Olson, Maplewood, MN (US); Boyce Moon, Ham Lake, MN (US)

(21) Appl. No.: 17/511,029

(22) Filed: Oct. 26, 2021

Related U.S. Application Data
(60) Provisional application No. 63/109,520, filed on Nov. 4, 2020.

(51) Int. Cl. A61B 18/14

(52) U.S. Cl. CPC A61B 18/1492; A61B 2018/126

(57) ABSTRACT

Systems and methods for ablating tissue are provided. An ablation system includes a catheter having a plurality of electrodes, and a controller coupled to the catheter. The controller is configured to select at least one pair of non-adjacent electrodes of the plurality of electrodes, and sequentially apply bipolar stimulation using the at least one selected pair of non-adjacent electrodes.

### CROSS-REFERENCE TO RELATED APPLICATIONS

This application claims priority to provisional application Ser. No. 63/109,520, filed Nov. 4, 2020, which is incorporated herein by reference in its entirety.

### FIELD OF THE DISCLOSURE

The present disclosure relates generally to tissue ablation systems. In particular, the present disclosure relates to performing tissue ablation using non-adjacent bipoles in a sequential delivery scheme.

### BACKGROUND

It is generally known that ablation therapy may be used to treat various conditions afflicting the human anatomy. One such condition in which ablation therapy finds a particular application in, for example, is the treatment of atrial arrhythmias. When tissue is ablated, or at least subjected to ablative energy generated by an ablation generator and delivered by an ablation catheter, lesions form in the tissue. Electrodes mounted on or in ablation catheters are used to create tissue necrosis in cardiac tissue to correct conditions such as atrial arrhythmia (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to trans-membrane potential, which opens the pores on the cell wall. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

At least some known ablation systems include a catheter having a variable diameter spiral, with a plurality of electrodes positioned on the variable diameter spiral. However, depending on the size of the spiral and the orientation of the catheter, electrodes with opposite polarities may overlap, potentially resulting in arcing or shunted current paths, which is generally undesirable.

### BRIEF SUMMARY OF THE DISCLOSURE

In one embodiment, the present disclosure is directed to an ablation system. The ablation system includes a catheter including a plurality of electrodes, and a controller coupled to the catheter. The controller is configured to select at least one pair of non-adjacent electrodes of the plurality of electrodes, and sequentially apply bipolar stimulation using the at least one selected pair of non-adjacent electrodes.

In another embodiment, the present disclosure is directed to a method for ablating tissue using a catheter including a plurality of electrodes. The method includes selecting, using a controller coupled to the catheter, at least one pair of non-adjacent electrodes of the plurality of electrodes, and sequentially applying bipolar stimulation using the at least one selected pair of non-adjacent electrodes.

In yet another embodiment, the present disclosure is directed to a controller for use in an ablation therapy system. The controller is coupled to a catheter including a plurality of electrodes, and the controller includes a memory device, and a processor coupled to the memory device, the processor configured to select at least one pair of non-adjacent electrodes of the plurality of electrodes, and control the catheter to sequentially apply bipolar stimulation using the at least one selected pair of non-adjacent electrodes.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1A is a schematic and block diagram view of a system for electroporation therapy.

FIGS. 1B and 1C are views of one embodiment of a distal spiral subassembly that may be used with the catheter shown in FIG. 1A.

FIGS. 2A and 2B illustrate lesion depths for different electrode configurations.

FIG. 3A illustrates a catheter having an odd number of electrodes.

FIG. 3B illustrates a lesion pattern generated using the catheter shown in FIG. 3A.

FIG. 4 is a flow diagram of a method for ablating tissue.

FIG. 5 is a block diagram illustrating different examples of selecting at least one pair of non-adjacent electrodes.

### DETAILED DESCRIPTION OF THE DISCLOSURE

The present disclosure provides systems and methods for ablating tissue. In one embodiment, the present disclosure is directed to an ablation system. The ablation system includes a catheter including a plurality of electrodes, and a controller coupled to the catheter. The controller is configured to select at least one pair of non-adjacent electrodes of the plurality of electrodes, and sequentially apply bipolar stimulation using the at least one selected pair of non-adjacent electrodes.

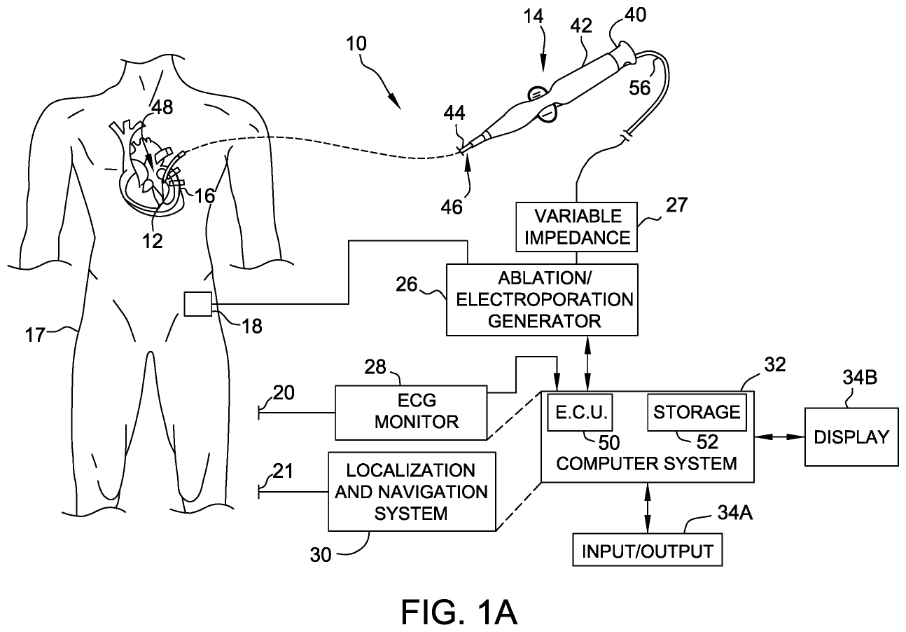

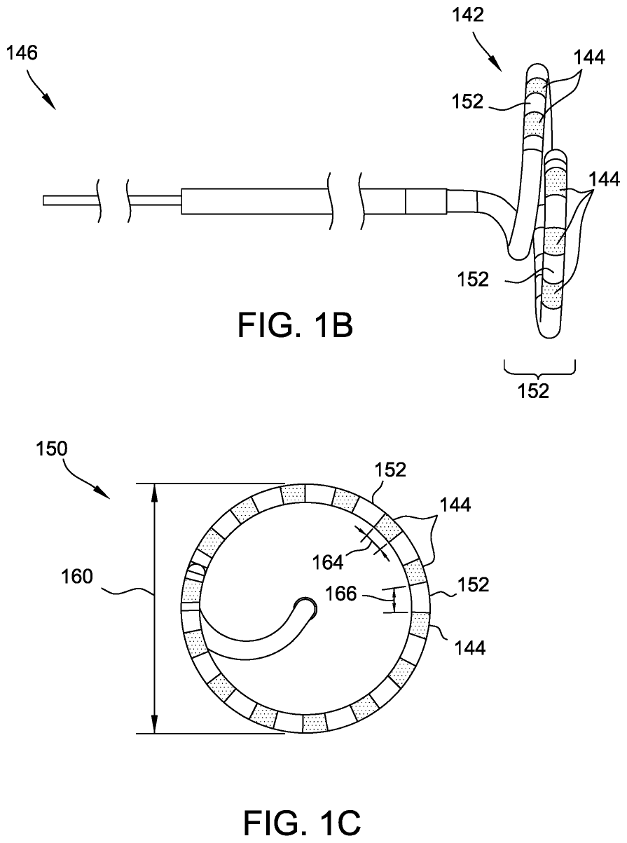

FIG. 1A is a block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 10 may be used for irreversible electroporation (IRE) to destroy tissue. In particular, system 10 may be used for electroporation-induced primary necrosis therapy, which refers to the effects of delivering electrical current in such a manner as to directly cause an irreversible loss of plasma membrane (cell wall) integrity leading to its breakdown and cell necrosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field in the form of short-duration pulses (e.g., having a 0.1 to 20 microsecond (μs) duration) between closely spaced electrodes capable of delivering an electric field strength of about 0.1 to 1.0 kilovolts/centimeter (kV/cm). System 10 may be used, for example, with a high output spiral catheter (See FIGS. 1B and 1C) for high output (e.g., high voltage and/or high current) electroporation procedures.

In one embodiment, all electrodes of the spiral catheter deliver an electric current simultaneously (while in other embodiments, stimulation sequentially switches between different electrode pairs, as described in detail herein). That is, the electrodes are electrically connected in parallel during the application. Delivering electric current simultaneously using a plurality of electrodes arranged in a circular fashion facilitates creating a sufficiently deep lesion for electroporation. To facilitate activating electrodes simultaneously, the electrodes may be switchable between being connected to a 3D mapping system and being connected to EP amplifiers. For a spiral catheter, as explained below, when the spiral diameter is minimized, multiple electrodes may overlap with one another.

Irreversible electroporation through a multi-electrode spiral catheter may enable pulmonary vein isolation in as few as one shock per vein, which may produce much shorter procedure times compared to sequentially positioning a radiofrequency (RF) ablation tip around a vein.

It should be understood that while the energization strategies are described as involving DC pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption through application of a high-voltage electric field. Thus, electroporation may avoid some possible thermal effects that may occur when using radio frequency (RF) energy. This “cold therapy” thus has desirable characteristics.

With this background, and now referring again to FIG. 1A, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues.

FIG. 1A further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation-induced primary necrosis therapy, generator 26 may be configured to produce an electric current that is delivered via electrode assembly 12 as a pulsed electric field in the form of short-duration DC pulses (e.g., a nanosecond to several milliseconds duration, 0.1 to 20 ms duration, or any duration suitable for electroporation) between closely spaced electrodes capable of delivering an electric field strength (i.e., at the tissue site) of about 0.1 to 1.0 kV/cm. The amplitude and pulse duration needed for irreversible electroporation are inversely related. As pulse durations are decreased, the amplitude must be increased to achieve electroporation.

Electroporation generator 26, sometimes also referred to herein as a DC energy source, is a monophasic electroporation generator 26 configured to generate a series of DC energy pulses that all produce current in the same direction. In other embodiments, electroporation generator is biphasic or polyphasic electroporation generator configured to produce DC energy pulses that do not all produce current in the same direction. In some embodiments, electroporation generator 26 is configured to output energy in DC pulses at selectable energy levels, such as fifty joules, one hundred joules, two hundred joules, and the like. Other embodiments may have more or fewer energy settings and the values of the available setting may be the same or different. For successful electroporation, some embodiments utilize the two hundred joule output level. For example, electroporation generator 26 may output a DC pulse having a peak magnitude from about 300 Volts (V) to about 3,200 V at the two hundred joule output level. In some embodiments, the peak magnitude may be even larger (e.g., on the order of 10,000 V). Other embodiments may output any other suitable positive or negative voltage.

In some embodiments, a variable impedance 27 allows the impedance of system 10 to be varied to limit arcing from the catheter electrode of catheter 14. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1A, as noted above, catheter 14 may include functionality for electroporation and in certain embodiments also an ablation function (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter. Handle 42 is also conventional in the art and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

In some embodiments, catheter 14 is a spiral catheter having catheter electrodes (not shown) distributed at the distal end of shaft 44. The diameter of the spiral may be variable. In some embodiments, the spiral catheter has a maximum diameter of about twenty-seven millimeters (mm). In some embodiments, the spiral diameter is variable between about fifteen mm and about twenty eight mm. Alternatively, the catheter may be a fixed diameter spiral catheter or may be variable between different diameters. In some embodiments, catheter 14 has fourteen catheter electrodes. In other embodiments, catheter 14 includes ten catheter electrodes, twenty catheter electrodes, or any other suitable number of electrodes for performing electroporation. In some embodiments, the catheter electrodes are ring electrodes, such as platinum ring electrodes. Alternatively, the catheter electrodes may be any other suitable type of electrodes, such as partial ring electrodes or electrodes printed on a flex material. In various embodiments, the catheter electrodes have lengths of 1.0 mm, 2.0 mm, 2.5 mm, and/or any other suitable length for electroporation.

Localization and navigation system 30 may be provided for visualization, mapping and navigation of internal body structures. Localization and navigation system 30 may include conventional apparatus known generally in the art (e.g., an EnSite Precision™ System, commercially available from Abbott Laboratories. and as generally shown with reference to commonly assigned U.S. Pat. No. 7,263,397 titled “Method and Apparatus for Catheter Navigation and Location and Mapping in the Heart,” the entire disclosure of which is incorporated herein by reference). In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that these system are examples only, and are not limiting in nature. Other technologies for locating/navigating a catheter in space (and for visualization) are known, including for example, the CARTO navigation and location system of Biosense Webster, Inc., the Rhythmia® system of Boston Scientific Schimed, Inc., the KODEX® system of Koninklijke Philips N.V., the AURORA® system of Northern Digital Inc., commonly available fluoroscopy systems, or a magnetic location system such as the gMPS system from Mediguide Ltd. In this regard, some of the localization, navigation and/or visualization system would involve a sensor be provided for producing signals indicative of catheter location information, and may include, for example one or more electrodes in the case of an impedance-based localization system, or alternatively, one or more coils (i.e., wire windings) configured to detect one or more characteristics of a magnetic field, for example in the case of a magnetic-field based localization system.

FIGS. 1B and 1C are views of one embodiment of a distal spiral subassembly 146 that may be used with catheter 14 in system 10. Those of skill in the art will appreciate that, in other embodiments, any suitable catheter may be used. Specifically, FIG. 1B is a side view of distal spiral subassembly 146 with a variable diameter spiral 150 at a distal end 142. FIG. 1C is an end view of variable diameter spiral 150 of distal spiral subassembly 146. Those of skill in the art will appreciate that, although the embodiments disclosed herein are discussed in the context of a variable diameter spiral, the methods and systems described herein (e.g., applying bipolar stimulation using non-adjacent electrode pairs) may be implemented using any suitable catheter (e.g., fixed loop catheters, linear catheters, etc.).

Variable diameter spiral 150 is selectively transitionable between an expanded (also referred to as “open”) diameter 160 (shown in FIG. 1C) and a retracted (also referred to as “closed”) diameter 160 (not shown). In the example embodiment, an expanded diameter 160 is twenty seven mm and a retracted diameter 160 is fifteen mm. In other embodiments, diameter 160 may be variable between any suitable open and closed diameters 160.

In the embodiment shown, variable diameter spiral 150 includes fourteen catheter electrodes 144 substantially evenly spaced around the circumference of variable diameter spiral 150 in the expanded configuration. In the retracted configuration, one or more of electrodes 144 may overlap, as described herein.

Catheter electrodes 144 are platinum ring electrodes configured to conduct and/or discharge electrical current in the range of one thousand volts and/or ten amperes. In other embodiments, variable diameter spiral 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode by an insulated gap 152. In the example embodiment, each catheter electrode 144 has a same length 164 (shown in FIG. 1C) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter spiral 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter spiral 150, while still allowing enough flexibility to allow variable diameter spiral 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter spiral 150 while also decreasing current density (by increasing the surface area) on electrodes 144, which may help prevent arcing during electroporation operations. Increasing length 164 too much, however, may prevent variable diameter spiral 150 from forming a smooth circular shape and may limit the closed diameter 160 of variable diameter spiral 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing during electroporation, and may result in larger additional system resistances needing to be added to prevent arcing. Moreover, in order to get a desired, even coverage about the circumference of variable diameter spiral 150, more catheter electrodes 144 may be needed if length 164 is decreased. Increasing the number of catheter electrodes 144 on variable diameter spiral 150 may prevent variable diameter spiral 150 from being able to be contracted to a desired minimum diameter 160.

Pulsed field ablation (PFA) has been shown to be an effective form of ablation for treatment of cardiac arrhythmias, particularly for instantaneous pulmonary vein isolation (PVI). PFA includes delivering high voltage pulses from electrodes disposed on a catheter (e.g., including variable diameter spiral 150). In PFA, for example, voltage amplitudes may range from about 300 V to at least 3,200 V (or even as large as on the order as 10,000 V), and pulse widths may from hundreds of nanoseconds to tens of milliseconds.

These electric fields may be applied between adjacent electrodes (in a bipolar approach) or between a one or more electrodes and a return patch (in a monopolar approach). There are advantages and disadvantages to each of these approaches (e.g., when using variable diameter spiral 150).

For example, regarding lesion contiguity, the monopolar approach has the potential to leave gaps in lesion coverage (referred to as dead zones) between electrodes where the field strength is low or zero, whereas the field strength in the bipolar approach generally prevents dead zones between electrodes.

For lesion size and proximity, the monopolar approach has a wider range of effect, and can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions. However, the monopolar approach may create larger lesions than are necessary, while the lesions generated using the bipolar approach may be more localized.

Due to a wider range of effect, the monopolar approach may cause unwanted skeletal muscle and/or nerve activation. In contrast, the bipolar approach has a constrained range of effect proportional to electrode spacing on the lead, and is less likely to depolarize cardiac myocytes or nerve fibers.

For the monopolar approach, only a single potential is applied in catheter wires and electrodes. Further, because all the electrodes are at the same polarity, the configuration is not susceptible to arcing (e.g., when using variable diameter spiral 150). In contrast, for the bipolar approach, the internal architecture of the catheter must be constructed to prevent arcing, as different electrodes are at different potentials. Further, with a catheter having a variable diameter spiral at the distal end (e.g., variable diameter spiral 150), depending on the size of the spiral and the orientation of the catheter, electrodes with opposite polarities may overlap, potentially resulting in arcing or shunted current paths, which is generally undesirable. Further, interleaved electrodes may interfere with signals used for tissue sensing.

When operating a catheter with a variable diameter spiral under the bipolar approach, it is desirable to assess whether electrodes on the catheter overlap with one another or are interleaved with one another, increasing the likelihood of arcing or shunted current paths. For example, when the diameter of variable diameter spiral 150 is reduced, one or more electrodes 144 may overlap or become interleaved with one another.

Accordingly, in one embodiment, to assess whether electrodes overlap or are interleaved, impedances between pairs of electrodes 144 can be measured while a low amplitude current is delivered between that pair of electrodes 144. This can be applied to each possible pair of electrodes 144, or only a subset of the possible pairs. For example, it generally is not physically possible for electrodes144 that are adjacent to one another along the length of variable diameter spiral 150 to be overlapping, as an insulated gap 152 separates adjacent electrodes 144, regardless of the diameter of variable diameter spiral 150. Accordingly, there is little need to assess whether adjacent electrodes 144 overlap (as it is physically impossible for them to do so).

This technique enables determining whether pairs of electrodes 144 may be shorting to one another or have a low-resistance current path between each other (indicating the electrodes 144 in the pair are overlapping or interleaved). Based on this, electrodes 144 with a high-impedance path between them may be selected for applying bipolar energy delivery.

Although this technique does enable detecting electrode shorting, it may not, by itself, solve the underlying problem. For example, a pair of electrodes 144 may be selected for stimulation based on the impedance measurements, but if the spiral is subsequently deflected or changes diameter, those selected electrodes 144 may begin to overlap or become interleaved. To mitigate this, the diameter and/or orientation of the spiral may be fixed, but that largely defeats the benefits of using a catheter having a variable diameter spiral. Accordingly, in at least some embodiments, bipolar energy delivery is performed sequentially using non-adjacent pairs of electrodes 144.

For a simultaneous energy delivery scheme, all electrodes 144 on variable diameter spiral 150 are energized at the same time. For example, all odd numbered electrodes 144 may be energized at a predetermined voltage, with all even numbered electrodes set as electrical returns (or vice versa).

In contrast, in sequential delivery, only a single pair of electrodes 144 or a limited number of pairs of electrodes 144 (i.e., not all electrodes) are energized at the same time. Once one or more pulses are delivered for the single pair or limited number of pairs, another set of electrode pairs is subsequently energized. This process may be repeated until all tissue proximate variable diameter spiral 150 has been covered.

If adjacent electrodes 144 (i.e., electrodes that are separated along variable diameter spiral by a single insulated gap 152) are energized sequentially, it may limit the potential for shorting. However, because of the proximity of the two adjacent electrodes 144, additional non-energized electrodes 144 proximate (e.g., at least partially overlapping with) the two adjacent electrodes 144 may offer a low impedance path, potentially causing arcing or limiting the amount of energy delivered to tissue.

Thus, in at least some embodiments, non-adjacent electrodes 144 are energized sequentially. This limits the potential for shorting, and also reduces the likelihood of a low impedance path existing between the pair of non-adjacent electrodes 144.

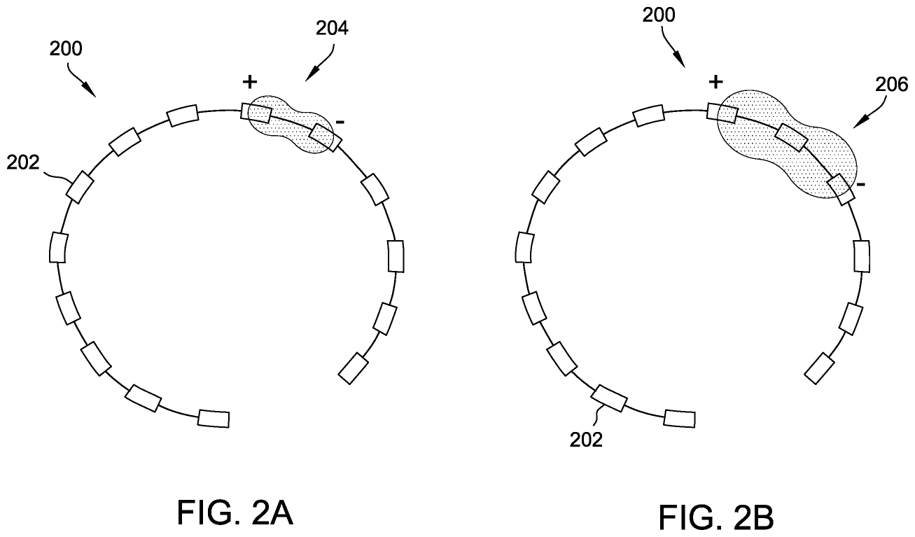

For example, assume there are N electrodes 144 on variable diameter spiral 150, numbered from 1 to N, with the most distal electrode being 1 and the most proximal electrode being N. Then, the following sequential stimulation sequence may be used: over a first time interval, energy is delivered from electrode 1 to electrode 3, over a second time interval subsequent to the first time interval, energy is delivered from electrode 2 to electrode 4, . . . , over a subsequent time interval, energy is delivered from electrode N−2 to electrode N. In this example, a single electrode 144 is skipped between bipoles (i.e., along the length of variable diameter spiral 150, a single non-energized electrode 144 separates the two electrodes 144 used for energy delivery). FIG. 2B (discussed below) shows an example of a single non-energized electrode separating two electrodes used for energy delivery.

In another example, two electrodes 144 may be skipped between bipoles. That is, the following stimulation sequence may be used: over a first time interval, energy is delivered from electrode 1 to electrode 4, over a second time interval subsequent to the first time interval, energy is delivered from electrode 2 to electrode 5, . . . , over a subsequent time interval, energy is delivered from electrode N−3 to electrode N. Those of skill in the art will appreciate that other non-adjacent sequential stimulation patterns may be employed within the spirit and scope of the disclosure.

For example, in some embodiments, pairs of non-adjacent electrodes 144 are selected by measuring impedances between pairs of non-adjacent electrodes 144 while delivering a low amplitude current between that pair of electrodes 144 (as described above), and selecting, for stimulation, pairs of non-adjacent electrodes 144 that have a relatively high measured impedance therebetween. In another embodiment, pairs of non-adjacent electrodes are selected based on user input (e.g., user input made using input/output mechanisms 34A and received at electronic control unit 50, both shown in FIG. 1A).

Because one or more electrodes 144 are skipped, employing sequential stimulation with non-adjacent electrodes 144 limits the ability to encounter a low impedance path between the energized electrodes, which may prevent arcing and facilitates ensuring that the therapeutic energy is not being lost to a low impedance path. Moreover, because the spacing between energized electrodes 144 is wider (as compared to energizing adjacent electrodes), a larger volume of tissue generally experiences an electric field strength sufficient for ablation. Accordingly, the wider bipole spacing makes non-adjacent stimulation schemes competitive with monopolar approaches with regards to lesion depth. Additionally, lesion depth may be controlled by selecting how many electrodes 144 should be skipped between the energized bipoles.

More specifically, a resultant voltage gradient field between two bipolar electrodes is three-dimensional, and is proportional to the distance between the bipoles. Thus, larger distances between the bipoles (in combination with larger applied voltages), generally result in a voltage gradient field having a larger radius, resulting in deeper lesions. For example, FIGS. 2A and 2B illustrate a catheter 200 having a plurality of electrodes 202. In FIG. 2A, the energized bipoles are two adjacent electrodes 202, generating a first voltage gradient field 204. In contrast, in FIG. 2B, the energized bipoles are non-adjacent electrodes 202 separated by an inactive electrode 202 (i.e., the energized bipoles are non-adjacent electrodes 202), generating a second voltage gradient field 206. As shown in FIGS. 2A and 2B, the second voltage gradient field 206 is larger than the first voltage gradient field 204, corresponding to greater lesion depth.

Notably, most of the field strength in the voltage gradient field is proximate the bipoles, such that beyond a certain separation distance, the resulting lesion will not be contiguous. Accordingly, to ensure a fully contiguous lesion set, all electrodes should be used as part of one or more active bipole pairs. It may also beneficial that each electrode serves as both positive and a negative side of the bipole over the stimulation sequence, as the field shape is asymmetric and stronger towards the center of the bipole.

In one embodiment, the number of skipped electrodes is determined based on a desired lesion depth. For example, a controller (e.g., implemented using computer system 32) coupled to the catheter may determine a target lesion depth (e.g., based on user input). Then, based on the determined target lesion depth, the controller selects, for stimulation, one or more non-adjacent electrode pairs that will facilitate achieving the target lesion depth.

Those of skill in the art will appreciate that many possible combinations of non-adjacent electrode pairs may be used in the spirit of the embodiments described herein. This includes, for example, energizing electrodes 144 that are positioned roughly 180° from one another around variable diameter spiral 150 (i.e., across from one another on variable diameter spiral 150). This pairing provides the largest bipole that can be formed within variable diameter spiral 150. For example, if variable diameter spiral 150 includes twelve electrodes, labeled 1 to 12 and arranged similar to the numbers on a clock, the following stimulation sequence could be utilized: over a first time interval, energy is delivered from electrode 12 to electrode 6, over a second time interval subsequent to the first time interval, energy is delivered from electrode 1 to electrode 7, over a third time interval, energy is delivered from electrode 2 to electrode 8, over a fourth time interval, energy is delivered from electrode 3 to electrode 9, over a fifth time interval, energy is delivered from electrode 4 to electrode 10, and over a sixth time interval, energy is delivered from electrode 5 to electrode 11.

Using the techniques described herein, sequential energization using non-adjacent bipoles can inherently overcome issues of electrode shorting. Further, as noted above, the increased distance between the bipoles facilitates creating deeper lesions, making lesion depth capabilities competitive with monopolar delivery. However, because the delivery is still bipolar, the applied electric field is limited to a local region, reducing the likelihood of adverse events due to non-targeted regions being exposed to relatively high voltage energies.

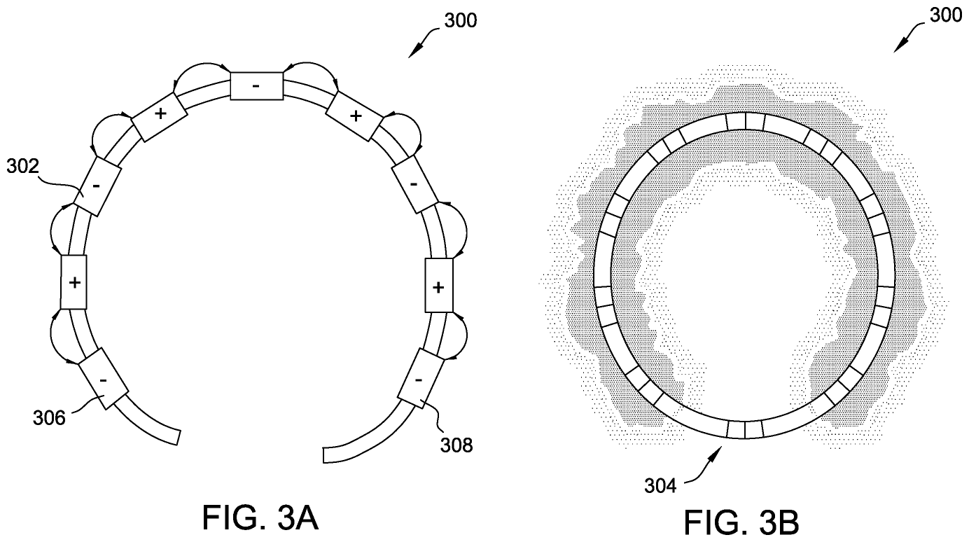

Yet another benefit of selecting arbitrary bipoles for energy delivery is the capability to bridge large gaps in catheter designs that have a relatively large distance between proximal and distal electrodes. This may be particularly useful when the catheter includes an odd number of electrodes. Specifically, in existing systems, when a spiral catheter includes an odd number of electrodes, the polarity of the distal and proximal electrodes are the same, resulting in an area of low field strength between the distal and proximal electrodes, and thus a gap in lesion coverage. For example, FIG. 3A illustrates a catheter 300 having an odd number of electrodes 302, with the polarity alternating between adjacent electrodes 302. As shown in FIG. 3B, a gap 304 in the applied electric field occurs between a proximal electrode 306 and a distal electrode 308, preventing contiguity of the lesion pattern.

This can be overcome in existing systems by rotating the catheter. However, the systems and methods described herein, which enable arbitrarily assigning polarity to electrodes on the catheter, generally allow for the creation of a contiguous lesion set without repositioning the catheter (with the caveat that large spaces between bipoles may result in a discontinuous lesion, as noted above).

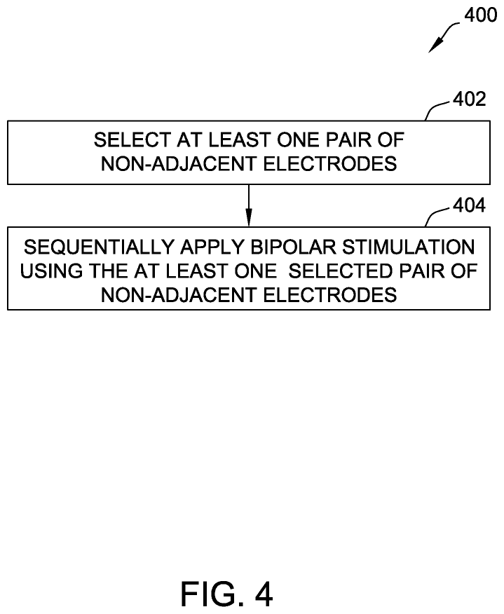

FIG. 4 is a flow diagram of one embodiment of a method 400 for ablating tissue. Method 400 may be performed, for example, using system 10 (shown in FIGS. 1A-1C). Method 400 includes selecting 402 at least one pair of non-adjacent electrodes. The pair of non-adjacent electrodes may be selected 402, for example, based on impedance measurements and/or user input. In some embodiments, the pair of non-adjacent electrodes may be selected 402 without relying on an impedance measurement (i.e., statically selected). Subsequently, bipolar stimulation is sequentially applied 404 using the at least one selected pair of non-adjacent electrodes.

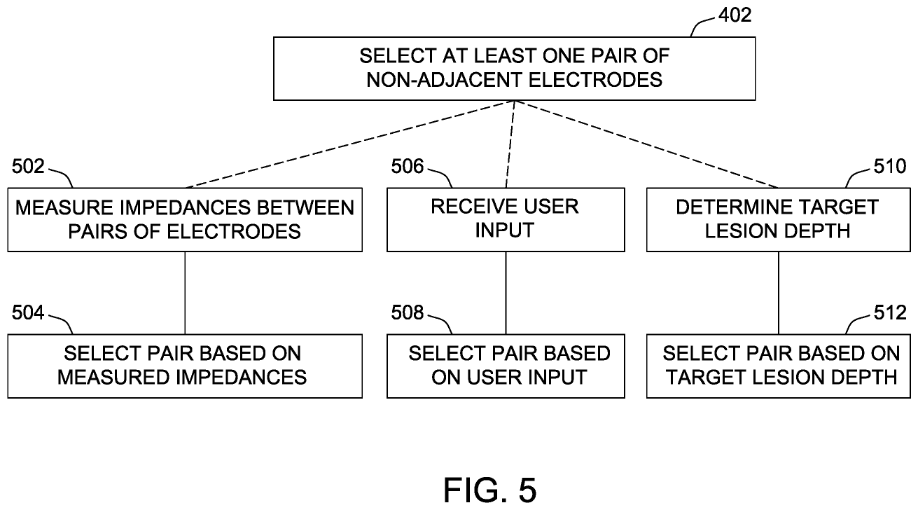

FIG. 5 is a block diagram illustrating different examples of selecting 402 at least one pair of non-adjacent electrodes. Those of skill in the art will appreciate that these examples are not mutually exclusive of one another, and two or more examples may be combined in a particular embodiment.

In one example, selecting 402 includes measuring 502 impedances between a plurality of different pairs of electrodes, and selecting 504 the at least one pair based on the measured impedances. In another example, selecting 402 includes receiving 506 a user input at a controller coupled to the catheter, and selecting 508 the at least one pair based on the received user input. In yet another example, selecting 402 includes determining 510 a target lesion depth (e.g., based on a user input), and selecting 512 the at least one pair based on the target lesion depth.

The systems and methods described herein are directed to ablating tissue. An ablation system includes a catheter having a plurality of electrodes, and a controller coupled to the catheter. The controller is configured to select at least one pair of non-adjacent electrodes of the plurality of electrodes, and sequentially apply bipolar stimulation using the at least one selected pair of non-adjacent electrodes.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An ablation system comprising:
a catheter comprising a plurality of electrodes; and
a controller coupled to the catheter, the controller configured to:
select at least one pair of non-adjacent electrodes of the plurality of electrodes; and
sequentially apply bipolar stimulation using the at least one selected pair of non-adjacent electrodes.

2. The ablation system of claim 1, wherein to select at least one pair of non-adjacent electrodes, the controller is configured to select a pair of electrodes that are separated by one electrode along a length of the catheter.

3. The ablation system of claim 1, wherein to select at least one pair of non-adjacent electrodes, the controller is configured to select a pair of electrodes that are separated by at least two electrodes along a length of the catheter.

4. The ablation system of claim 1, wherein the catheter comprises a variable diameter spiral.

5. The ablation system of claim 1, wherein to select at least one pair of non-adjacent electrodes, the controller is configured to:
measure impedances between a plurality of different pairs of electrodes of the plurality of electrodes; and
select the at least one pair of electrodes based on the measured impedances.

6. The ablation system of claim 1, wherein to select at least one pair of non-adjacent electrodes, the controller is configured to select the at least one pair of electrodes based on a user input received at the controller.

7. The ablation system of claim 1, wherein to select at least one pair of non-adjacent electrodes, the controller is configured to select the at least one pair of electrodes to achieve a target lesion depth.

8. A method for ablating tissue using a catheter including a plurality of electrodes, the method comprising:
selecting, using a controller coupled to the catheter, at least one pair of non-adjacent electrodes of the plurality of electrodes; and
sequentially applying bipolar stimulation using the at least one selected pair of non-adjacent electrodes.

9. The method in accordance with claim 8, wherein selecting at least one pair of non-adjacent electrodes comprises selecting a pair of electrodes that are separated by one electrode along a length of the catheter.

10. The method in accordance with claim 8, wherein selecting at least one pair of non-adjacent electrodes comprises selecting a pair of electrodes that are separated by at least two electrodes along a length of the catheter.

11. The method in accordance with claim 8, wherein the catheter is a variable diameter spiral, and wherein selecting at least one pair of non-adjacent electrodes comprises selecting a pair of electrodes that are substantially across from one another on the variable diameter spiral.

12. The method in accordance with claim 8, wherein selecting at least one pair of non-adjacent electrodes comprises:
measuring impedances between a plurality of different pairs of electrodes of the plurality of electrodes; and
selecting the at least one pair of electrodes based on the measured impedances.

13. The method in accordance with claim 8, wherein selecting at least one pair of non-adjacent electrodes comprises selecting the at least one pair of electrodes based on a user input received at the controller.

14. The method in accordance with claim 8, wherein selecting at least one pair of non-adjacent electrodes comprises selecting the at least one pair of electrodes to achieve a target lesion depth.

15. A controller for use in an ablation therapy system, the controller coupled to a catheter including a plurality of electrodes, wherein the controller comprises:
a memory device; and
a processor coupled to the memory device, the processor configured to:
select at least one pair of non-adjacent electrodes of the plurality of electrodes; and
control the catheter to sequentially apply bipolar stimulation using the at least one selected pair of non-adjacent electrodes.

16. The controller in accordance with claim 15, wherein to select at least one pair of non-adjacent electrodes, the controller is configured to select a pair of electrodes that are separated by one electrode along a length of the catheter.

17. The controller in accordance with claim 15, wherein to select at least one pair of non-adjacent electrodes, the controller is configured to select a pair of electrodes that are separated by at least two electrodes along a length of the catheter.

18. The controller in accordance with claim 15, wherein the controller is configured to select at least one pair of non-adjacent electrodes on a variable diameter spiral of the catheter.

19. The controller in accordance with claim 15, wherein the controller is configured to select at least one pair of non-adjacent electrodes based on a user input received at the controller.

20. The controller in accordance with claim 15, wherein the controller is configured to select at least one pair of non-adjacent electrodes to achieve a target lesion depth.
